(19) United States
(12) Patent Application Publication
(10) Pub. No.: US 2008/0292713 A1
(43) Pub. Date: Nov. 27, 2008

(54) Respirable Powders

(75) Inventors: Peter Craig Seville, Birmingham (GB); Tristan Paul Learoyd, Birmingham (GB)

Correspondence Address: MICHAEL BEST & FRIEDRICH LLP, ONE SOUTH PINCKNEY STREET, P O BOX 1806, MADISON, WI, 53701, US

(73) Assignee: ASTON UNIVERSITY, Birmingham (GB)

(21) Appl. No.: 12/095,046

(22) PCT Filed: Aug. 25, 2006
(86) PCT No.: PCT/GB06/03184

§ 371 Date: May 27, 2008

(30) Foreign Application Priority Data
Nov. 28, 2005 (GB) 0524194.8

(51) Int. Cl. A61K 9/14; A61K 31/135; A61P 11/00

(52) U.S. Cl. 424/499; 424/501; 514/653

(57) ABSTRACT

A spray dried dispersible powdered composition suitable for inhalation by a human subject, which composition comprises: a) at least one active agent suitable for treating a condition in said subject by inhalation; b) a hydrophobic amino acid; and c) a pharmaceutically acceptable biodegradable polymer.

The present invention relates to spray-dried respirable powders for sustained drug delivery.

Over the years, certain drugs have been sold in compositions suitable for forming a drug dispersion for oral inhalation (pulmonary delivery) to treat various conditions in humans. Such pulmonary drug delivery compositions are designed to be delivered by inhalation of a drug dispersion by the patient so that the active drug within the dispersion can reach the lung. Drugs deposited in the alveolar region are absorbed across the alveolar epithelium and subsequently distributed throughout the body via the blood circulation. In contrast, locally deposited drugs (i.e. deposition in the central region of the lung, such as the bronchioles) are absorbed at the site of deposition and exert their pharmacological action on receptors located in the central bronchioles (e.g. salbutamol interacts with β2 adrenoceptors).

Pulmonary drug delivery can itself be achieved by different approaches, including liquid nebulizers, aerosol-based metered dose inhalers (MDI's), and dry powder dispersion devices. Much redevelopment of pMDI systems has taken place, using hydrofluoroalkane (HFA) propellants in place of the traditional chlorofluorocarbon (CFC) propellants, although the marked differences in the physicochemical properties of the replacement propellants has been, and still is, causing significant reformulation difficulties.

Traditionally, dry powders have been formulated as micronised drug blended with a coarse carrier particle, typically lactose. More recently, there has been increased interest in the use of spray-dried powders in order to achieve better performance in terms of decreased oropharyngeal deposition and increased fine particle fraction (FPF).

The use of amino acids as aerosolisation enhancers in such spray-dried respirable powders has been previously disclosed (Li, H.-Y., et al., Journal of Drug Targeting, 11 (7), 425-432 (2003); Najafabadi, A. R., et al., International Journal of Pharmaceuticals, 285, 97-108 (2004); Li, H.-Y., et al., The Journal of Gene Medicine, 7, 343-353 (2005)). For example, leucine has been shown to increase the fine particle fraction of a spray-dried respirable powder which would lead to decreased oropharyngeal deposition.

Across the spectrum of drug delivery methods, including spray-dried respirable powders, biodegradable polymers have been used to provided sustained delivery of a drug active. For example, polylactide co-glycolide has been used to produce spray-dried particles of betamethasone which had sustained release properties (Chaw, C. S., et al., J. Microencapsulation, 20(3), 349-359 (2003)). Likewise, chitosan and hydroxypropyl methylcellulose (HPMC) have been used to generate spray-dried powders of carbamazepine that exhibit sustained release properties (Filipovic-Grcic, J., et al., J. Pharm. Pharmacol., 55, 921-931 (2003)).

The present inventors have now discovered that, contrary to expectations, certain amino acids can be used to enhance the dispersibility of spray-dried respirable powders containing a pharmaceutically acceptable biodegradable polymer.

Accordingly, the first aspect of the present invention provides a spray dried dispersible powdered composition suitable for inhalation by a human subject, which composition comprises:
a) at least one active agent suitable for treating a condition in said subject by inhalation;
b) a hydrophobic amino acid; and
c) a pharmaceutically acceptable biodegradable polymer.

Such particles have been shown to retain their key property of sustained release of the active agent, whilst having improved dispersibility and thereby improving the deposition pattern of the powder following aerosolisation.

The composition of the present invention may further comprise:
d) a pharmaceutically acceptable bulking agent comprising a carbohydrate.

A second aspect of the present invention provides the use of a composition of the first aspect in the manufacture of a medicament for treating a condition in a human that is susceptible to treatment by oral inhalation, the treatment comprising inhaling an aerosolized composition.

A third aspect of the present invention provides a method for preparing a spray-dried dispersible powder according to the first aspect of the invention.

### DEFINITIONS

The following terms which are used throughout the description of the present invention are defined as detailed below.

The term “powder” or “powdered” refers to a composition that consists of finely dispersed solid particles that are relatively free flowing and capable of being dispersed in an inhalation device and subsequently inhaled by a subject so that the particles reach the lungs to permit penetration into the central and/or alveolar region of the lung. Thus, the powder is administrable by inhalation therapy and is said to be “respirable” and suitable for pulmonary delivery. In general, the average particle size is less than about 10 μm in diameter and the particle shapes may be irregular, uniform or mixed. Preferably, the average particle size is less than about 7.5 μm and more preferably less than about 5.0 μm. Usually the particle size distribution is between about 0.1 μm and about 5 μm in diameter, particularly about 2 μm to about 5 μm.

The term “dry” means that the powder composition has a moisture content such that the particles are readily dispersible in an inhalation device to form an aerosol. This moisture content is generally trapped within the particle matrix and is generally below about 30% by weight (% w/w) water.

The term “fine particle fraction (FPF)” means the proportion of the total dose less the 5 μm in aerodynamic diameter. A standard measurement of fine particle fraction is described hereinafter, as the fraction of particles being less that 5 μm in aerodynamic diameter when assessed by a cumulative plot from data derived from the Andersen Cascade Impactor.

The term “dispersibility” means the degree to which a powder composition can be dispersed (i.e. suspended) in a current of air so that the dispersed particles can be respired or inhaled into the lungs of a subject. For example, a powder composition that is only 10% dispersible means that only 10% of the mass of finely-divided particles making up the composition can be suspended for oral inhalation into the lungs; 50% dispersibility means that 50% of the mass can be suspended. A standard measurement of dispersibility is described hereinafter as the fraction of total dose emitted from a hydroxyl-propyl methylcellulose (HPMC) size 3 capsule by a spinhaler device by 5 litres of ambient air at a rate of 60 L/min.

The term “therapeutically effective amount” is the amount of an active agent present in the powder composition that is needed to provide the desired level of the active agent to a subject to be treated to give the anticipated physiological response. This amount is determined for each active agent on a case-by-case basis.

The term “physiologically effective amount” is that amount delivered to a subject to give the desired palliative or curative effect. This amount is specific for each active agent and its ultimately approved dosage level.

The term “pharmaceutically acceptable” refers to an excipient, whether a carrier or the protein used to improve dispersibility, that can be taken into the lungs with no significant adverse toxicological effects on the lungs.

### Compositions

The component of the composition that is an active agent includes any agent that is useful for treating a human subject by inhalation therapy.

Administration of the active agent exhibits a beneficial effect on the subject in that there is a palliative or curative affect on the subject's condition. Thus, the subject may be suffering from bronchial asthma or related corticosteroid-responsive bronchospastic states, hayfever, an inflamed condition, endometriosis, prostatic cancer, a bacterial infection, viral infection, or the like. In addition, the subject may be suffering from a condition that would require the administration of a nucleic acid complex of DNA or RNA material for gene therapy treatment or a treatment of a condition responsive to treatment by an interferon such as hepatitis B and C, Hairy Cell Leukemia, chronic hepatitis Non A, Non B/C, Kaposis Sarcoma, multiple sclerosis, chronic granulomatous disease, and the like. Thus, the types of active agents suitable for use in the composition include steroids (e.g. dexamethasone, triamcinolone, beclomethasone, beclomethasone dipropionate, fluocinolone, fluocinonide, flunisolide, flunisolide hemihydrate, and the like), bronchodilators (e.g. adrenalin, isoproterenol, metaproterenol, terbutaline and its salts, isoetharine, albuterol and its salts, pirbuterol and its salts, bitolterate, and the like), mast cell inhibitors (cromolyn sodium, and the like), antibiotics (e.g. pentamidine), low molecular weight polypeptides such as LHRH and its derivatives (LHRH, nafarelin, goserelin, leuprolide, and the like), high molecular weight polypeptides such as interferon or rhuIL-I receptor, and the like. Also an active agent that is an RNA or DNA sequence that is useful for gene therapy may be employed as part of the composition of this invention. Generally the amount of active agent present in the composition will vary between about 0.1% w/w to about 50% w/w, preferably from about 0.5% w/w to about 20% w/w, and most preferably from about 2% w/w to about 10% w/w.

Suitable bulking agents for use in the present invention are carbohydrates, and, in particular, mono- and polysaccharides. Representative monosaccharides include carbohydrate excipients such as dextrose (anhydrous and the monohydrate; also referred to as glucose and glucose monohydrate), galactose, mannitol, D-mannose, sorbitol, sorbose and the like. Representative disaccharides include, but are not limited to, lactose, maltose, sucrose, trehalose. Representative trisaccharides include, but are not limited to, raffinose. Other carbohydrate excipients include cyclodextrins such as 2-hydroxypropyl-β-cyclodextrin.

The amount of optional bulking agent that is useful in the composition of this invention is an amount that serves to dilute the active agent to a concentration at which the active agent can provide the desired beneficial palliative or curative results while at the same time minimising any adverse side effects that might occur from too high a concentration. Thus, for an active agent that exhibits a high physiological activity, more of the bulking agent will be used, whereas for an active agent that exhibits a low physiological activity, less of the bulking agent will be used. In some compositions, it will not be necessary to employ a bulking agent, depending on the concentrations of the active agent, the hydrophobic amino acid and the pharmaceutically acceptable biodegradable polymer employed. In general, the amount of bulking agent in the composition will be between about 0.1% w/w and 99.9% w/w of the total composition.

Suitable hydrophobic amino acids, which are those amino acids that are more hydrophobic in nature, include naturally occurring amino acids which are non-polar, e.g. alanine, isoleucine, leucine, methionine, phenylalanine, proline, tryptophan and valine. These amino acids are generally available from commercial sources that provide pharmaceutical grade products.

Suitable pharmaceutically acceptable biodegradable polymers include chitosan, polyethylene glycol, hydroxypropyl methylcellulose (HPMC), polyacrylic acid, polyvinyl pyrrolidone, polylactide co-glycolide (PLGA) and methacrylic acid. Preferably, hydrophobic polymers, such as chitosan, are employed. These polymers are generally available from commercial sources that provide pharmaceutical grade products.

In addition to the components listed above, the composition of this invention may contain other pharmaceutically-acceptable excipients that may be used to stabilize the composition or make it more compatible with the unit dosage form from which it is delivered. Such excipients include, for example, buffers such as citrate, phosphate or acetate. These components, if present, will usually form no more than 5% w/w of the composition.

The composition of this invention typically will be delivered from a unit dosage receptacle containing an amount that will be sufficient to provide the desired physiological effect upon inhalation by a subject in need thereof. The amount will be dispersed in a chamber that has an internal volume sufficient to capture substantially all of the powder dispersion resulting from the unit dosage receptacle.

The unit dosage will be from about 2 mg of powder to about 50 mg of powder, preferably about 15 mg to about 30 mg. About 25 mg of powder per unit dosage is quite effective. The preferred unit dosage receptacle is a hard gelatin or HPMC (hydroxyl-propyl methycellulose) capsule. The general process of preparing such capsules is generally known to one of skill in the art from such publications as Remington's Pharmaceutical Sciences (21^{st} Edition) or other similar publications. The capsule will be of a suitable size to accommodate the amount of powder required and for operation in a suitable inhalation device, such as a Spinhaler.

### Administration

Another aspect of this invention is a method of administering a therapeutically effective amount of a powdered composition of this invention to a human subject in need thereof by dispersing said powdered composition as an aerosol into a chamber having a delivery outlet suitable for inhalation therapy, e.g., a mouthpiece and having said subject inhale, preferably orally, said dispersed powder into the subject's lungs.

Various suitable devices and methods of inhalation which can be used to administer particles to a patient's respiratory tract are known in the art. For example, suitable inhalers are described in U.S. Pat. No. 4,995,385, U.S. Pat. No. 4,069,819 and U.S. Pat. No. 5,997,848. Other examples include, but are not limited to, the Spinhaler (Fisons, Loughborough, U.K.), Rotahaler (Glaxo-Wellcome, Research Triangle Technology Park, N.C.), FlowCaps (Hovione, Loures, Portugal), Inhalator (Boehringer-Ingelheim, Germany), the Aerolizer (Novartis, Switzerland), the Diskhaler (Glaxo-Wellcome, RTP, NC) and others, such as known to those skilled in the art. Such devices are reviewed by Atkins, P. J. in Respir Care., 50(10), 1304-12 (2005), which is incorporated herein by reference.

Preferably, the particles are administered as a dry powder via a unit-dose dry powder inhaler, such as a Spinhaler In practice, a preferred unit dosage of powdered composition of this invention of about 4 mg to about 50 mg is subjected to conditions discussed hereinafter to aerosolize the powder so that a standing cloud or aerosol dispersion is created in a suitable chamber and a subject then orally inhales the dispersion into the subject's lungs.

### Preparation of Compositions

There are two main methods for preparing compositions of the present invention.

The first method is aimed at producing particles that are essentially homogenous and that release at least one active agent over a sustained period. The second method is aimed at producing particles which have a heterogeneous structure, and that could be used to release two or more active agents over different time periods. The second method could, however, also be used to release a single active over a sustained period or with differing release rates.

### First Method

The first method comprises spray-drying a solution or suspension of all the components of the composition. The solvent used should be pharmaceutically acceptable, and may be water, organic solvents or a mixture of the two. In particular, preferred organic solvents are pharmaceutically acceptable alcohols, and more preferably ethanol. A preferred solvent is a mixture of ethanol in water, where the ethanol is between 10% by volume (% v/v) to 50% v/v. More preferably, the ethanol is between 20% v/v and 40% v/v, and is most preferably about 30% % v/v of ethanol in water.

In the preparation of the mixture for spray-drying, a solution or suspension is prepared by dissolving or dispersing the components of the composition in the solvent. The proportion of the components of the composition in the mixture should be consistent with the proportions that are desired in the resultant powdered composition. In general, the mixture will have a total powder mass of between about 0.1 g and 10 g per 100 mL mixture (i.e. 0.1 to 10% w/v). Preferably, the total powder mass will be at least 0.5 g per 100 mL mixture (i.e. 0.5% w/v) and less than 5 g per 100 mL mixture (i.e. 5% w/v). More preferably, the total powder mass will be about 2 g per 100 mL mixture (i.e. 2% w/v).

Generally, the mixture for spray-drying is formed by mixing appropriate amounts of the components of the composition with an appropriate volume of solvent, and stirring until all the materials have dissolved/dispersed. Usually, it is sufficient to prepare the solution/suspension at a temperature of about 20° C. to 30° C., more preferably at ambient temperature, although gentle heat may be employed to facilitate dissolution of the components, provided this does not adversely affect the activity or the stability of the components; many components, particularly the active agent, of the present invention are know to be heat-sensitive.

### Second Method

The second method comprises spray-drying an emulsion of the components of the composition. Without wishing to be bound by theory, it is thought that the different phases of the emulsion form different layers within the resultant powder particles. These different layers can be used to allow delivery of two or more active agents over different time periods. As mentioned above, the second method can also be used produce particles that release a single active over a sustained period.

In the simplest case, the emulsion is an oil-in-water (o/w) single emulsion, with the oil phase containing one or more hydrophobic active agents (e.g. beclometasone dipropionate) and the biodegradable polymer, and the water phase containing the hydrophobic amino acid and optionally a bulking agent. Optionally, one or more hydrophilic active agents can also be incorporated in the water phase, although these active agents would not undergo sustained release.

A more complex case involves the preparation of a water-in-oil-in-water (w/o/w) double emulsion, whereby a primary water-in-oil (w/o) emulsion is formed from a water phase which may contain one or more hydrophilic active agents and an oil phase containing a biodegradable polymer and optionally one or more hydrophobic active agents. There must be at least one active agent in the primary emulsion. This primary emulsion is subsequently emulsified into a further water phase containing the hydrophobic amino acid and optionally a bulking agent. Optionally, one or more hydrophilic active agents can also be incorporated in this water phase, although these active agents would not undergo sustained release.

It would be possible to continue to build more complex emulsions, with active agents incorporated in various phases, although complex emulsions containing more than 5 phases are likely to be pharmaceutically impractical. In all cases, the final phase should be a water phase containing the hydrophobic amino acid and optionally a bulking agent.

In general, the emulsion will have a total powder mass (of the components (a) to (d)) of between about 0.1 g and 10 g per 100 mL emulsion (i.e. 0.1 to 10% w/v). Preferably, the total powder mass will be at least 0.5 g per 100 mL emulsion (i.e. 0.5% w/v) and less than 5 g per 100 mL emulsion (i.e. 5% w/v). More preferably, the total powder mass will be about 0.5 g to 1 g per 100 mL emulsion.

### Spray-Drying

The mixture for spray-drying produced under the first or second methods is subsequently spray-dried under conditions sufficient to produce a dispersible powdered pharmaceutical composition having an aerodynamic diameter of less than about 10 μm. This method consists of bringing together the mixture described above with a sufficient volume of hot air to produce evaporation and drying of the liquid droplets. In general, the liquid mixture is sprayed into a current of warm air that evaporates the solvent and conveys the dried product to a collector. The spent air is then exhausted with the solvent. Alternatively, inert gases, such as nitrogen, can be used instead of air. The resultant spray-dried powdered particles are approximately spherical in shape. A further discussion of spray-drying can be found in Chapter 37 of Remington's at pages 702-706, which is incorporated herein by reference.

It is found that the process of this invention works particularly well using a Buchi mini spray-dryer apparatus having a serial number of 290. Generally the inlet temperature and the outlet temperature of the spray dry equipment are not critical but will be of such a level to provide the desired particle size and to result in a product that has the desired activity of the active agent. The inlet temperature thus may be between temperatures of 100° C. to about 200° C., preferably from 150° C. to 200° C. and more preferably about 180° C. The flow rate of the feed which is used in the spray drying equipment generally will be about 2 ml per minute to about 5 ml per minute, preferably 3 ml per minute to 4 ml per minute, and more preferably about 3.2 ml per minute. The atomizer air flow rate will vary between values of 500 litres per minute to about 700 litres per minute, preferably about 600 liters per minute. Secondary drying is not generally needed, but may be employed.

### Further Preferences

Any of the preferences below may be combined with one another, or with any of the those expressed above, which may also be combined with one another.

For particles produced of the present invention, preferred active agents include the hydrophilic agents and p agonists terbutaline sulfate and salbutamol sulfate and the hydrophobic corticosteroid beclometasone diproprionate.

A preferred loading of active agents in particles produced by the first method is from 2 to 6% w/w, and more preferably about 4% w/w. A preferred loading of active agents in particles produced by the second method is from 6 to 10% w/w, and more preferably about 8% w/w. If two or more active agents are loaded in different layers, the amount of the active agent in those layers containing active agent is preferably from 6 to 10% w/w, and more preferably about 8% w/w.

In particles produced by the first method, the pharmaceutically acceptable polymer may preferably be chitosan, or its derivatives, and it may be used in any of its varying molecular weights. The polymer is preferably incorporated at 25 to 50% w/w.

In particles produced by the second method, the pharmaceutically acceptable polymer may preferably be polylactide co-glycolide, and in particular 75:25 PLGA. The polymer is preferably incorporated at 2.5 to 10%, and is preferably used at the highest concentration possible to maximize duration of sustained release.

In particles of the present invention, the preferred amino acid is leucine. The amino acid is preferably included at as high proportion as possible. It is preferably present in an amount up to 50% w/w, more preferably up to 40% w/w. The lower limit may be as low as 1% w/w, but is preferably 5% w/w, 10% w/w or even 20% w/w.

Preferred bulking agents in the present invention include disaccharides, and in particular lactose.

In the second method, an emulsifier, such as PVA, may be used to help form emulsions where water is the primary phase. In these case, the amount of emulsifier is preferably as small as possible to enable the formation of a homogenous emulsion.

### FIGURES

FIG. 1 shows the dissolution profile of an comparative embodiment.

FIG. 2 shows the relative amounts of deposition in the Anderson Cascade Impactor test of a comparative embodiment.

FIG. 3a shows the relative amounts of deposition in the Anderson Cascade Impactor test of an embodiment of the invention, and

FIG. 3b shows the dissolution profile of the same embodiment.

FIGS. 4a to 4d are scanning electron micrographs of embodiments of the invention.

FIG. 5 shows the dissolution profile of another embodiment of the invention.

FIG. 6 shows the dissolution profile of a further embodiment of the invention.

FIG. 7 shows the dissolution profile of a further embodiment of the invention.

FIG. 8 shows the dissolution profile of a further embodiment of the invention.

### EXAMPLES

### Techniques

Laser diffraction: approximately 5 mg of the spray-dried powders were suspended in hexane and the suspension ultrasonicated (Soniprep 150; Curtin Matheson Scientific Ltd, Houston, Tex., USA) for 30 s. The particle size of the sample was measured by laser diffraction (Mastersizer; Malvern Instruments, Malvern, UK) using a 100 mm focal length lens. Each sample was measured in triplicate. The data obtained were expressed in terms of the particle diameter 50% of the volume distribution (d[v,50]).

Scanning electron microscopy (SEM): Spray-dried powders were mounted on double-faced adhesive tape and sputter-layered with gold under partial vacuum in an EMScope® SC500 sputter machine. Representative scanning electron micrographs were taken using a Cambridge Instruments Sterioscan 90, with image processing by a Pixie 8 Image Processor.

Tapped density: The tapped density of the spray-dried powders was determined by tapped density measurements using a tamping voltameter (USP2 Tapped Density Assessor, Copley Scientific Ltd, UK). Measurements were performed in triplicate.

Primary aerodynamic diameter (dₐₑ): Theoretical estimates of particle primary aerodynamic diameter were derived from the particle sizing and tapped density data, according to Equation 1:

$${d}_{ae} = d \sqrt{\frac{p}{{p}_{1}}} \qquad Equation 1$$

where d is the median particle diameter (d[v,50]), p is the powder tapped density and p₁=1 g cm⁻³.

Andersen Cascade Impactor: Pre-weighed capsules filled to within +/−2.5% of a specified dry mass (25 mg) were packed into Hydroxypropyl methylcellulose (HPMC) size 3 capsules and fixed to a Spinhaler dry powder inhaler device. The Spinhaler was expelled into an ACI at 60 Lmin⁻¹ using a Copley flowmeter DFM2 in two five second bursts thirty seconds apart. The ACI Plates were coated with 1% silicon oil in hexane to minimize bounce. The filter stage was covered by a Whatman 934-AH glass filter paper. After expiration empty capsule was then gravimetrically assessed for emitted dose expressed as a percentage of the original pre-weighed dose. The process repeated in triplicate. After each cycle the ACI was dissembled and the stages washed with aqueous methanol (30% v/v), collected, and assessed using High Performance Liquid Chromatography (HPLC). For HPLC a Waters Associates chromatography pump ran at 1 ml/min, Waters Associates Wisp 710B, Severn Analytical SA6503 programmable absorbance detector were combined with JCL6000 for windows for 2.0. The column used was a Spher OD22 SU. For terbutaline hemisulphate, salbutamol sulfate and beclometasone diproprionate detection a mobile phase of aqueous methanol (70:30) was used at 278 μm, 275 μm and 250 μm wavelengths respectively. Using the cut off diameters of each stage on the ACI, a cumulative curve was constructed from the filter stage to the throat/pre-separator. At a flow rate of 60 Lmin⁻¹, the effective cut-off diameters of the modified ACI are: Stage −1, 8.6 μm; Stage −0, 6.5 μm; Stage 1, 4.4 μm; Stage 2, 3.2 μm; Stage 3, 1.9 μm; Stage 4, 1.2 μm; Stage 5, 0.55 μm; and Stage 6, 0.26 μm. 2.6.3. The fine particle dose (FPD), defined as the mass of drug less than 5 μm, was calculated by interpolation from a plot of cumulative mass vs. effective cut-off diameter of the respective stages. The fine particle fraction (FPF) was calculated as the ratio of FPD to total loaded dose, expressed as a percentage and corrected for actual drug content in each powder. The mass median aerodynamic diameter (MMAD) of the powders was also derived, defined as the particle size at the 50% mark of a plot of cumulative fraction vs. effective cut-off diameter. The dispersibility of the powder was calculated as the percentage of the total powder mass loaded into the capsule that is emitted from the capsule during aerosolisation, determined by weighing the capsule and its contents before and after aerosolisation. A powder with a high dispersibility will show a more beneficial deposition profile, and exhibit greater deposition in the target region of the lung, than a powder that displays poor dispersibility.

Dissolution tests: Dissolution tests were carried out using a Hanson Research SR6 SR116 flask (Hanson, USA), Calveda Dissolution Model 6SG and Model 7ST (Calveda, USA), and Sotax AT7 (Sotax, UK) USP2 dissolution apparatus modified with attached paddle baskets containing 200 mg of pre-wetted powder were used to monitor release profiles of the spray dried blends. The baskets were rotated at 50 rpm in 200 ml of 37° C. phosphate buffer solution. Readings where taken at selected time intervals and subsequently tested for drug content using HPLC. Powders that displayed drug release over a timescale of at least one hour were considered to exhibit sustained release properties.

### First Method

### Materials

Model active agents, the hydrophilic β2 agonists terbutaline (Sigma Aldrich Ltd, UK) and salbutamol (Sigma Aldrich Ltd, UK) and the hydrophobic corticosteroid beclometasone (Sigma Aldrich Ltd, UK), all used in the treatment of acute and chronic respiratory distress, were selected to assess the release of the spray dried formulations. The amino acid leucine (Sigma Aldrich Ltd, UK) was incorporated into the blends as an aerosolisation enhancer. Chitosan (low, medium and/or high molecular weight, Sigma Aldrich Ltd, UK), a water insoluble polysaccharide polymer derived from crustacean shell was incorporated into the blend for controlled release. Lactose (Fisher Scientific Ltd, UK), a disaccharide, was used as a bulking agent/diluent.

### Comparative Example 1

This comparative example describes the effect of spray-drying a composition of an active agent (salbutamol sulphate), a hydrophobic amino acid (leucine) and a carbohydrate bulking agent (lactose) in the absence of a polymer, as required by the present invention.

160 mg salbutamol sulphate, 720 mg leucine and 1.12 g lactose were dissolved in 100 mL of 30% v/v aqueous ethanol. The resulting solution was processed into a spray-dried powder using a Buchi B-290 mini spray-dryer using the following standard operating conditions: atomizer air flow rate 600 liters per minute; flow rate of the feed 3.2 ml per minute; inlet temperature 180° C. The resultant powder contained 8% w/w salbutamol sulphate, 36% w/w leucine and 56% w/w lactose.

| | | | | | Dissolution |
|---|---|---|---|---|---|
| | | Dispersi- | | | profile |
| | Yield | bility | MMAD | FPF | (Drug release |
| Preparation | (%) | (%) | (μm) | (%) | after time, t) |
| Comparative | 50 | 96.0 | 4.6 ± 0.5 | 29.7 ± 5.1 | 97.7% ± 2.1 |
| Example 1 | | | | | after 2 minutes |

The dissolution profile is shown in FIG. 1. This comparative example shows that exclusion of a polymer from the composition results in a respirable powder that displays no sustained drug release profile.

### Comparative Example 2

This comparative example describes the effect of spray-drying a composition of an active agent (terbutaline sulphate), a polymer (chitosan) and a carbohydrate bulking agent (lactose) in the absence of a hydrophobic amino acid, as required by the present invention.

40 g medium MW chitosan was added to 30 mL glacial acetic acid and stirred rigorously to form a gel. Distilled water was slowly added with stirring to a final volume of 1 L. 80 mg terbutaline sulphate and 920 mg lactose were dissolved in 45 mL distilled water. 25 mL of the chitosan gel was slowly added with stirring, followed by the addition of 30 mL ethanol. The resultant mixture was spray-dried using the standard operating conditions described above. The resultant powder contained 4% w/w terbutaline sulphate, 50% w/w medium MW chitosan and 46% w/w lactose.

| | | | | | Dissolution |
|---|---|---|---|---|---|
| | | Dispersi- | | | profile |
| | Yield | bility | MMAD | FPF | (Drug release |
| Preparation | (%) | (%) | (μm) | (%) | after time, t) |
| Comparative | 69 | 95.6 | 5.4 ± 0.3 | 20.7 ± 0.1 | 90.8 ± 7.3% |
| Example 2 | | | | | after 2 hours |

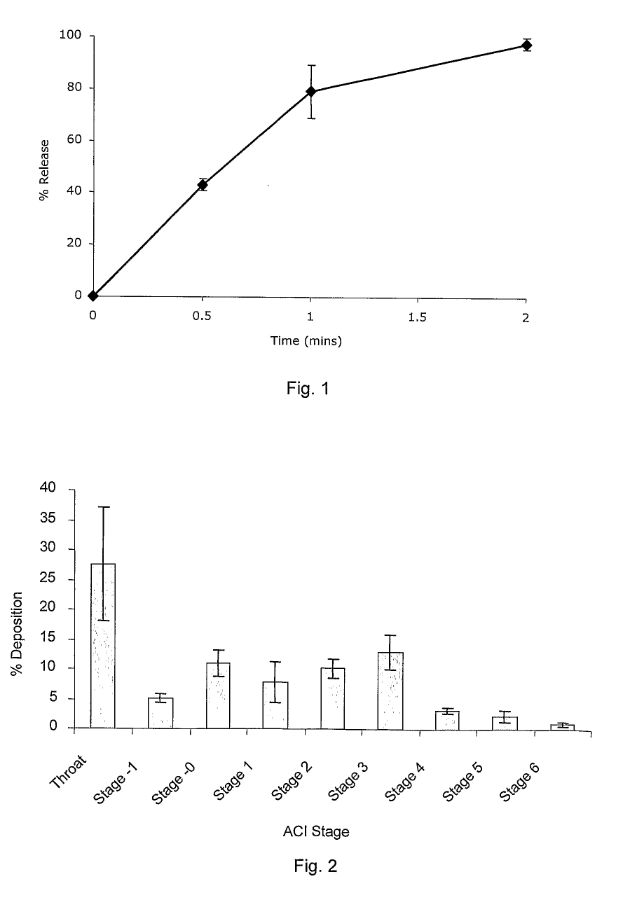

This comparative example shows that exclusion of the hydrophobic amino acid from the composition results in a poorly respirable powder with high deposition on the upper stages of the ACI (equivalent to oropharyngeal deposition), as shown in FIG. 2. However, inclusion of the hydrophobic polymer decreases the rate of dissolution of the drug, resulting in a sustained drug release profile.

### Example 1

This example of the present invention describes the effect of spray-drying a composition of an active agent (terbutaline sulphate), a polymer (chitosan), a hydrophobic amino acid (leucine) and a carbohydrate bulking agent (lactose).

15 g high MW chitosan was added to 3 mL glacial acetic acid and stirred rigorously to form a gel. Distilled water was slowly added with stirring to a final volume of 550 mL. 80 mg terbutaline sulphate, 720 mg leucine and 200 mg lactose were dissolved in 33 mL distilled water. 37 mL of the chitosan gel was slowly added with stirring, followed by the addition of 30 mL ethanol. The resultant mixture was spray-dried using the standard operating conditions described above. The resultant powder contained 4% w/w terbutaline sulphate, 36% w/w leucine, 50% w/w high MW chitosan and 10% w/w lactose.

| | | | | | Dissolution |
|---|---|---|---|---|---|
| | | Dispersi- | | | profile |
| | Yield | bility | MMAD | FPF | (Drug release |
| Preparation | (%) | (%) | (μm) | (%) | after time, t) |
| Example 1 | 59 | 95.3 | 4.8 ± 0.4 | 36.7 ± 1.4 | 100.0 ± 4.7% |
| | | | | | after 2 hours |

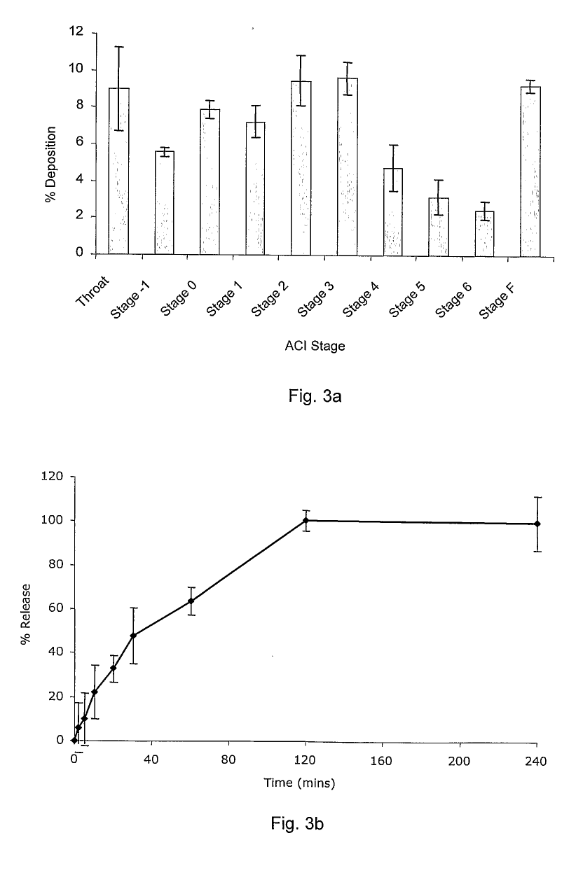

This example illustrates that spray-drying a composition that includes both a hydrophobic amino acid and a polymer results in a spray-dried powder that exhibits high respirability, i.e. shows less deposition on the upper stages of the ACI, as shown in FIG. 3a, and a sustained drug release profile, as displayed in FIG. 3b.

### Example 2

This example describes the effect of spray-drying a composition of an active agent (terbutaline sulphate), a polymer (chitosan) and a carbohydrate bulking agent (lactose) with variable amounts of a hydrophobic amino acid (leucine).

Compositions were prepared as described in Example 1, with the following changes to the proportion of leucine and lactose:
Example 2a: 120 mg leucine, 800 mg lactose
Example 2b: 240 mg leucine, 680 mg lactose
Example 2c: 360 mg leucine, 560 mg lactose
Example 2d: 720 mg leucine, 200 mg lactose

The resultant powders contained 4% w/w terbutaline sulfate, 50% w/w high MW chitosan, 6-36% w/w (6, 12, 18, 36% w/w) leucine and 40-10% w/w (40, 34, 28, 10% w/w) lactose.

| | | | | | Dissolution |
|---|---|---|---|---|---|
| | | Dispersi- | | | profile |
| | Yield | bility | MMAD | FPF | (Drug release |
| Preparation | (%) | (%) | (μm) | (%) | after time, t) |
| Example 2a | 77 | 97.8 | 6.0 ± 0.3 | 18.5 ± 3.3 | 89.1 ± 2.0% |
| | | | | | after 2 hours |
| Example 2b | 69 | 98.1 | 5.9 ± 0.4 | 23.9 ± 1.2 | 89.5 ± 4.1% |
| | | | | | after 2 hours |
| Example 2c | 77 | 92.6 | 6.7 ± 0.9 | 25.8 ± 1.0 | 92.0 ± 6.9% |
| | | | | | after 2 hours |
| Example 2d | 59 | 95.3 | 4.8 ± 0.4 | 36.7 ± 1.4 | 100.0 ± 4.7% |
| | | | | | after 2 hours |

This example illustrates that increasing the proportion of the hydrophobic amino acid in the composition increases the fraction of the powder that is respirable, whilst at the same time preserving the sustained drug release profile of the powder.

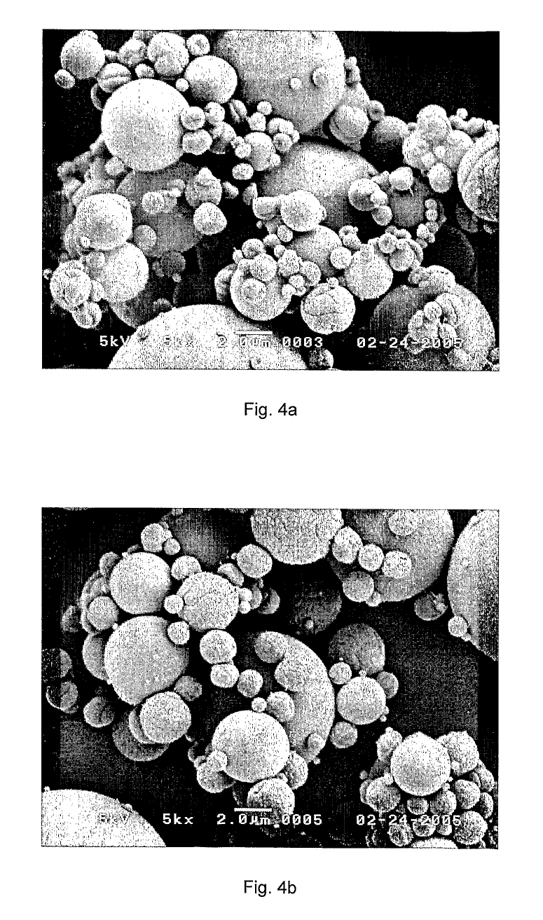

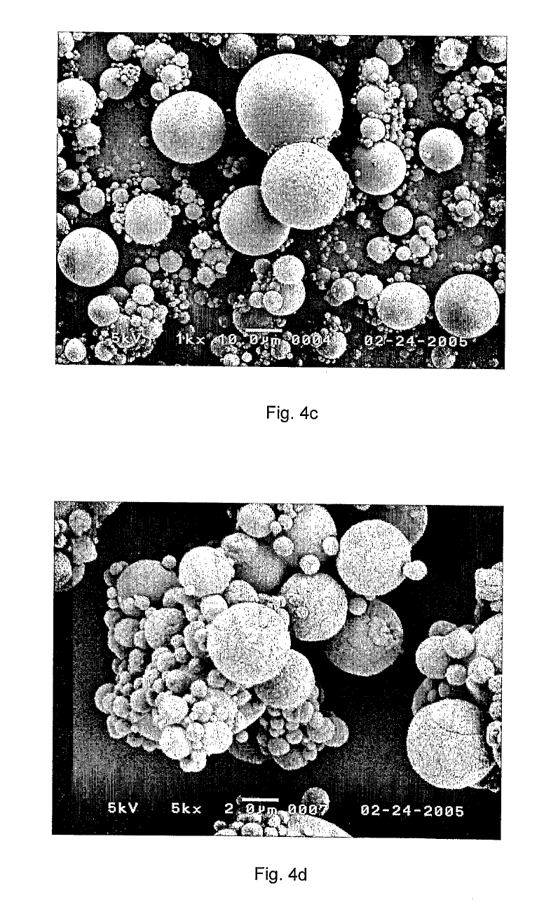

Scanning electron micrographs of the powder of Example 2b are shown as FIGS. 4a and 4b, and of the powder of Example 2c as FIGS. 4c and 4d.

### Example 3

This example describes the effect of spray-drying a composition of an active agent (terbutaline sulphate), a hydrophobic amino acid (leucine) and a carbohydrate bulking agent (lactose) with a polymer of different MW (medium and high MW chitosan).

Compositions were prepared as described above, using the following polymer:
Example 3a: 1 g medium MW chitosan
Example 3b: 1 g high MW chitosan
Example 3c: 0.5 g medium MW chitosan plus 0.5 g high MW chitosan

The resultant powders contained:
Example 3a: 4% w/w terbutaline sulphate, 36% w/w leucine, 10% w/w lactose and 50% w/w medium MW chitosan
Example 3b: 4% w/w terbutaline sulphate, 36% w/w leucine, 10% w/w lactose and 50% w/w high MW chitosan
Example 3c: 4% w/w terbutaline sulphate, 36% w/w leucine, 10% w/w lactose, 25% w/w medium MW chitosan and 25% w/w high MW chitosan

| | | | | | Dissolution | |
|---|---|---|---|---|---|---|
| | | Dispersi- | | | profile | |
| | Yield | bility | MMAD | FPF | (Time for 80% | |
| Preparation | (%) | (%) | (μm) | (%) | drug release) | |
| Example 3a | 56 | 96.4 | 6.4 ± 0.2 | 32.3 ± 2.1 | 60 | minutes |
| Example 3b | 59 | 95.3 | 4.8 ± 0.4 | 36.7 ± 1.4 | 110 | minutes |
| Example 3c | 62 | 97.4 | 5.1 ± 0.1 | 39.1 ± 0.4 | 80 | minutes |

This example illustrates the ability to tailor the rate of drug release through manipulation of the molecular weight of the polymer employed.

### Comparative Example 3

This comparative example describes the effect of spray-drying a composition of an active agent (terbutaline sulphate), a hydrophobic amino acid (leucine) and a carbohydrate bulking agent (lactose) with a polymer (polyethylene glycol: PEG) which falls outside the scope of the present invention.

80 mg terbutaline sulphate, 120 mg leucine, 200 mg PEG 400 and 1.6 g lactose were dissolved in 100 mL of 30% v/v aqueous ethanol. The resulting solution was processed into a spray-dried powder using a Buchi B-290 mini spray-dryer using the standard operating conditions. The resultant powder contained 4% w/w terbutaline sulphate, 6% w/w leucine, 10% PEG 400 and 80% w/w lactose.

| | | Dispersi- | | | |
|---|---|---|---|---|---|
| | Yield | bility | MMAD | FPF | Dissolution |
| Preparation | (%) | (%) | (μm) | (%) | profile |
| Comparative | 57 | 40 | 6.3 ± 1.9 | 4.7 ± 4.6 | — |
| Example 3 | | | | | |

This example illustrates the importance of the use of a hydrophobic polymer (e.g. chitosan) in the composition rather than a hydrophilic polymer (e.g. PEG). SEM further supported the ACI data by confirming particle sizes and revealing increased aggregation overtime.

### Example 4

This example shows the effect of spray-drying a composition of a hydrophilic active agent (4% W/W salbutamol sulphate), a hydrophobic amino acid (36% w/w leucine) and a carbohydrate bulking agent (10% w/w lactose) with a polymer of different MW (25% w/w medium MW chitosan and 25% w/w high MW chitosan).

| | | | | | Dissolution |
|---|---|---|---|---|---|
| | | Dispersi- | | | profile |
| | Yield | bility | MMAD | FPF | (Time for 80% |
| Preparation | (%) | (%) | (μm) | (%) | drug release) |
| Example 4 | 69.4 | 96.9 | 4.5 ± 0.5 | 47.5 ± 3.1 | 80 mins |

### Example 5

This example describes the effect of spray-drying a composition of a hydrophobic active agent (4% w/w beclometasone dipropionate), a hydrophobic amino acid (36% w/w leucine) and a carbohydrate bulking agent (10% w/w lactose) with a polymer of different MW (25% w/w medium MW chitosan and 25% w/w high MW chitosan)

| | | | | | Dissolution |
|---|---|---|---|---|---|
| | | Dispersi- | | | profile |
| | Yield | bility | MMAD | FPF | (Time for 80% |
| Preparation | (%) | (%) | (μm) | (%) | drug release) |
| Example 5 | 35.9 | 98.1 | 5.8 ± 0.1 | 33.0 ± 2.4 | 360 mins |

Examples 4 and 5 demonstrate the ability of the formulation to be applied to active agents with very different physical and chemical characteristics and achieve highly respirable powders with sustained release.

### Second Method

### Materials

Model active agents, terbutaline and salbutamol and beclometasone were selected to assess the release of the spray dried formulations. The amino acid leucine was incorporated into the blends as an aerosolisation enhancer. Polylactide co-glycolide 75:25 (PLGA) (Sigma Aldrich Ltd, UK) was employed as a hydrophobic erosion mediated polymer. Polyvinyl alcohol (PVA) (Sigma Aldrich Ltd, UK) was incorporated in to the secondary emulsion as a suspending agent. Lactose, a disaccharide, was used as a bulking agent/diluent.

### Comparative Example 4

This comparative example describes the effect of including dual layers of release by spray-drying a composition of a polymer (PLGA), a hydrophobic amino acid (leucine) and a carbohydrate bulking agent (lactose) as a double emulsion as above with salbutamol sulphate included in the tertiary water phase, not in the inner phase as required by the present invention.

0.4 mL water was vortex-mixed with 0.1 mL Span 80 (Sigma Aldrich Ltd, UK) and a solution of 200 mg PLGA in 12 mL chloroform to make a primary w/o emulsion. This primary emulsion was subsequently homogenized using a flat blade homogenizer at 1600 rpm with 288 mL of 10% w/v PVA aqueous solution containing 720 mg leucine, 160 mg salbutamol and a bulking agent of 920 mg lactose to form a w/o/w double emulsion containing salbutamol in the outer phase.

The resultant emulsion was spray-dried using the standard operating conditions described above.

| | Dispersi- | | | Dissolution profile |
|---|---|---|---|---|
| | bility | MMAD | FPF | (100% drug release |
| Preparation | (%) | (μm) | (%) | after time, t) |
| Comparative | 94.3 | 5.3 ± 0.3 | 25.5 ± 1.6 | 5 min |
| example 4 | | | | |

As indicated by the dissolution profile, inclusion of the drug in the outer phase results in a powder that exhibits rapid drug release, with no sustained release properties. This comparative example illustrates the need for active agents to be positioned in an earlier phase of the emulsion to display a high degree of sustained release.

### Example 6

This example of the present invention describes the effect of spray-drying a composition of an active agent (salbutamol sulphate), a polymer (PLGA), a hydrophobic amino acid (leucine) and a carbohydrate bulking agent (lactose) when formulated by the second method.

A solution of 160 mg salbutamol sulphate in 0.4 mL water was vortex-mixed with 0.1 mL Span 80 and a solution of 200 mg PLGA in 12 mL chloroform to make a primary w/o emulsion. This primary emulsion was subsequently homogenized using a flat blade homogenizer at 1600 rpm with 288 mL of 10% w/v PVA aqueous solution containing 720 mg leucine and a bulking agent of 920 mg lactose to form a w/o/w double emulsion containing salbutamol in the inner water phase.

The resultant emulsion was spray-dried using the standard operating conditions described above.

| | Dispersi- | | | Dissolution profile |
|---|---|---|---|---|
| | bility | MMAD | FPF | (90% Drug release |
| Preparation | (%) | (μm) | (%) | after time, t) |
| Example 6 | 95.9 | 6.1 ± 0.2 | 39.9 ± 2.6 | 7 days |

This example illustrates that spray-drying an emulsion that includes both a hydrophobic amino acid and polymer results in a spray-dried powder that exhibits high respirability and a sustained drug release profile when the active agent is present in the primary phase.

### Example 7

This example describes the effect of including dual layers of release by spray-drying a composition of an active agent (salbutamol sulphate), a polymer (PLGA), a hydrophobic amino acid (leucine) and a carbohydrate bulking agent (lactose) as a double emulsion as above with salbutamol in both the inner and outer water phases.

A solution of 160 mg salbutamol sulphate in 0.4 mL water was vortex-mixed with 0.1 mL Span 80 and a solution of 200 mg PLGA in 12 mL chloroform to make a primary w/o emulsion. This primary emulsion was subsequently homogenized using a flat blade homogenizer at 1600 rpm with 288 mL of 10% w/v PVA aqueous solution containing 720 mg leucine, 160 mg salbutamol and a bulking agent of 760 mg lactose to form a w/o/w double emulsion containing salbutamol in both the inner and outer water phases.

The resultant emulsion was spray-dried using the standard operating conditions described above.

| | Dispersi- | | | Dissolution profile | Dissolution profile |
|---|---|---|---|---|---|
| | bility | MMAD | FPF | (50% Drug release | (90% Drug release |
| Preparation | (%) | (μm) | (%) | after time, t) | after time, t) |
| Example 7 | 87.0 | 5.6 ± 0.2 | 34.1 ± 1.5 | 10 mins | 7 days |

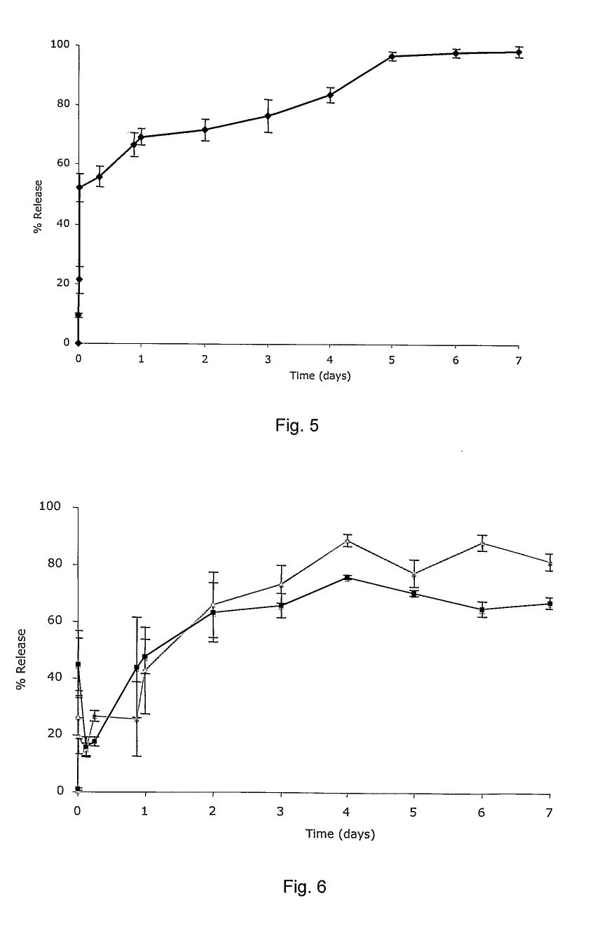

This example demonstrates a dual release profile by the positioning of the active agent in various phases of the emulsion, with a fast burst-release profile observed for the salbutamol contained in the outermost layer, and a sustained release profile observed for the salbutamol contained in the innermost layer, as shown in FIG. 5.

### Example 8

This example describes the effect of including dual layers of release by spray-drying a composition of two active agents (salbutamol sulphate and beclometasone dipropionate), a polymer (PLGA), a hydrophobic amino acid (leucine) and a carbohydrate bulking agent (lactose) as a double emulsion with salbutamol in the inner water phase and beclometasone dipropionate incorporated in the oil phase.

A solution of 160 mg salbutamol sulphate in 0.4 mL water was vortex-mixed with 0.1 mL Span 80 and a solution of 200 mg PLGA and 160 mg beclometasone dipropionate in 12 mL chloroform to make a primary w/o emulsion. This primary emulsion was subsequently homogenized using a flat blade homogenizer at 1600 rpm with 288 mL of 10% w/v PVA aqueous solution containing 720 mg leucine and a bulking agent of 760 mg lactose to form a w/o/w double emulsion containing salbutamol in the inner water phase and beclometasone dipropionate in the oil phase.

The resultant emulsion was spray-dried using the standard operating conditions described above.

| | Dispersi- | | | Dissolution profile | Dissolution profile |
|---|---|---|---|---|---|
| | bility | MMAD | FPF | (75% salbutamol release | (75% BDP release |
| Preparation | (%) | (μm) | (%) | after time, t) | after time, t) |
| Example 8 | 78.3 | 5.5 ± 0.6 | 37.8 ± 4.5 | 4 days | 4 days |

This example illustrates that a hydrophilic drug (salbutamol) incorporated into the inner water phase and a hydrophobic drug (BDP) incorporated into the oil phase both undergo similar sustained release profiles, as shown in FIG. 6 (⋄—BDP; ▪—salbutamol).

### Example 9

This example describes the effect of increasing PLGA polymer concentration by spray-drying a composition of an active agent (salbutamol sulphate), a polymer (PLGA), a hydrophobic amino acid (leucine) and a carbohydrate bulking agent (lactose) as a double emulsion as described above with salbutamol in the inner water phase.

Compositions were prepared as described in Example 6, with the following changes to the proportion of PLGA and lactose:
Example 9a: 50 mg PLGA, 1.07 g lactose
Example 9b: 100 mg PLGA, 1.02 g lactose
Example 9c: 150 mg PLGA, 970 mg lactose
Example 9d: 200 mg PLGA, 920 mg lactose

| | Dispersi- | | | Dissolution profile |
|---|---|---|---|---|
| | bility | MMAD | FPF | (80% Drug release |
| Preparation | (%) | (μm) | (%) | after time, t) |
| Example 9a | 91.7 | 5.2 ± 1.5 | 26.8 ± 5.2 | 4 days |
| Example 9b | 96.7 | 5.7 ± 0.2 | 31.35 ± 2.3 | 4 days |
| Example 9c | 97.4 | 5.8 ± 0.3 | 29.5 ± 1.6 | 6 days |
| Example 9d | 95.9 | 6.1 ± 0.2 | 40.0 ± 2.6 | 7 days |

This example shows the importance of the hydrophobic polymer PLGA on duration of release from the primary phase; increasing the proportion of PLGA in the emulsion results in a more sustained release profile exhibited by the powder.

### Example 10

This example describes the effect of including dual layers of release by spray-drying a composition of two active agent (salbutamol sulphate and beclometasone dipropionate), a polymer (PLGA), a hydrophobic amino acid (leucine) and a carbohydrate bulking agent (lactose) as a double emulsion as above with salbutamol in both the inner and outer water phases and beclometasone in the oil phase.

A solution of 160 mg salbutamol sulphate in 0.4 mL water was vortex-mixed with 0.1 mL Span 80 and a solution of 200 mg PLGA and 160 mg beclometasone dipropionate in 12 mL chloroform to make a primary w/o emulsion. This primary emulsion was subsequently homogenized using a flat blade homogenizer at 1600 rpm with 288 mL of 10% w/v PVA aqueous solution containing 720 mg leucine, 160 mg salbutamol and a bulking agent of 760 mg lactose to form a w/o/w double emulsion containing salbutamol in both the inner and outer water phases and beclometasone in the oil phase. The resultant emulsion was spray-dried using the standard operating conditions described above.

| | Dispersi- | | | Dissolution profile | Dissolution profile | Dissolution profile | Dissolution profile |
|---|---|---|---|---|---|---|---|
| | bility | MMAD | FPF | (50% salbutmol release | (50% BDP release | (95% salbutmol release | (95% BDP release |
| Preparation | (%) | (μm) | (%) | after time, t) | after time, t) | after time, t) | after time, t) |
| Example 10 | 87.1 | 5.3 ± 2.0 | 50.2 ± 8.1 | 6 hours | 2 days | 4 days | 5 days |

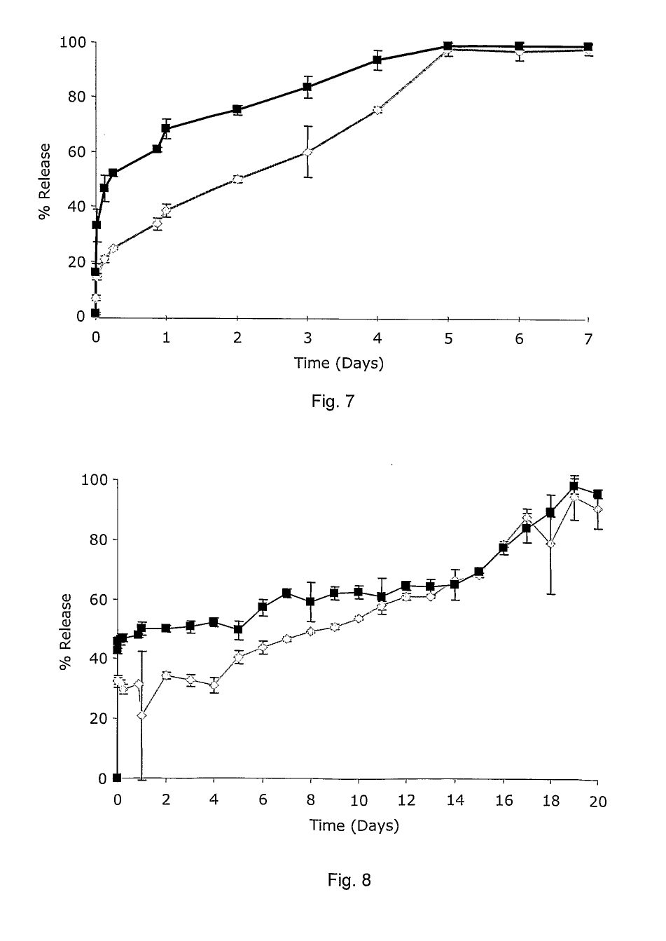

This example demonstrates a dual release profile by the positioning of the active agent in various phases of the emulsion, with a faster burst-release profile observed for the salbutamol contained in the outermost layer, and a sustained release profile observed for the salbutamol contained in the innermost layer and beclometasone contained in the middle layer, as shown in FIG. 7 (▪salbutamol; ♦BDP).

### Variation on Second Method

### Example 11

In this example, PLGA 50:50 was employed as the hydrophobic erosion mediated polymer, in place of PLGA 75:25, and chitosan was incorporated into the secondary emulsion as a suspending agent, in place of PVA.

A solution of 80 mg salbutamol sulphate in 1 mL water was vortex-mixed with 0.02 mL Span 80 and a solution of 200 mg PLGA 50:50 and 80 mg beclometasone dipropionate in 3 mL chloroform to make a primary w/o emulsion. This primary emulsion was subsequently homogenized using a flat blade homogenizer at 1600 rpm with 25 mL low molecular weight chitosan gel (4% w/v chitosan in 3% v/v glacial acetic acid) containing 560 mg leucine and 80 mg salbutamol, diluted to 100 mL with aqueous ethanol (30% v/v) to form w/o/w emulsions containing salbutamol in the inner and outer aqueous phases and BDP in the oil phase. The resultant emulsion was spray-dried using the standard operating conditions described above.

| | Dispersi- | | | Dissolution profile | Dissolution profile | Dissolution profile | Dissolution profile |
|---|---|---|---|---|---|---|---|
| | bility | MMAD | FPF | (50% salbutmol release | (50% BDP release | (95% salbutmol release | (95% BDP release |
| Preparation | (%) | (μm) | (%) | after time, t) | after time, t) | after time, t) | after time, t) |
| Example 11 | 96.6 | 2.7 ± 0.4 | 58.9 ± 1.8 | 1 day | 9 days | 19 days | 19 days |

This example demonstrates a dual release profile by the positioning of the active agent in various phases of the emulsion, with a faster burst-release profile observed for the salbutamol contained in the outermost layer, and a sustained release profile observed for the salbutamol contained in the innermost layer and beclometasone contained in the middle layer, as shown in figure a (▪ salbutamol; ♦BDP). This example also demonstrates that extremely long sustained release can be achieved through the use of PLGA 50:50 rather than PLGA 75:25, enabling the development of tailored release profiles through the selection of formulation excipients.

1. A spray dried dispersible powdered composition suitable for inhalation by a human subject, which composition comprises:
a) at least one active agent suitable for treating a condition in said subject by inhalation;
b) a hydrophobic amino acid; and
c) a pharmaceutically acceptable hydrophobic biodegradable polymer.

2. A composition according to claim 1, which further comprises:
d) a pharmaceutically acceptable bulking agent comprising a carbohydrate.

3. A composition according to claim 1, wherein the at least one active agent is selected from the group consisting of: steroids, bronchodilators, mast cell inhibitors, antibiotics, low molecular weight polypeptides and high molecular weight polypeptides.

4. A composition according to claim 3, wherein the at least one active agent is selected from terbutaline sulfate, salbutamol sulfate and beclometasone diproprionate.

5. (canceled)

6. A composition according to claim 1, wherein the bulking agent is selected from mono- and polysaccharides.

7. (canceled)

8. A composition according claim 1, wherein the hydrophobic amino acids is selected from alanine, isoleucine, leucine, methionine, phenylalanine, proline, tryptophan and valine.

9. A composition according to claim 8, wherein the hydrophobic amino acid is leucine.

10. A composition according to claim 1, wherein the pharmaceutically acceptable hydrophobic biodegradable polymer is selected from chitosan, hydroxypropyl methylcellulose (HPMC), polyvinyl pyrrolidone and poly-lactide co-glycolide (PLGA).

11-12. (canceled)

13. A unit dosage receptacle containing an amount of a composition according to claim 1, that will be sufficient to provide the desired physiological effect upon inhalation by a subject in need thereof.

14. A method for preparing a spray-dried dispersible powdered composition according to claim 1, wherein a solution or suspension of all the components of the composition in a pharmaceutically acceptable solvent is spray-dried.

15. A method according to claim 10, wherein the solvent is selected from water, ethanol, or a mixture of the two.

16. A method according to claim 14, wherein the mixture to be spray-dried will have a total powder mass of between about 0.1 g and 10 g per 100 mL mixture.

17. A method for preparing a spray-dried dispersible powdered composition according to claim 1, wherein an emulsion of all the components of the composition is spray-dried.

18. A method according to claim 17, wherein the emulsion is an oil-in-water single emulsion, with the oil phase containing one or more hydrophobic active agents and the hydrophobic biodegradable polymer, and the water phase containing the hydrophobic amino acid and a bulking agent, if present.

19. (canceled)

20. A method according to claim 17, wherein the emulsion is a water-in-oil-in-water double emulsion, whereby a primary water-in-oil (w/o) emulsion containing at least one active agent is formed from a water phase which may contain one or more hydrophilic active agents and an oil phase containing a hydrophobic biodegradable polymer and optionally one or more hydrophobic active agents, and said primary emulsion is subsequently emulsified into a further water phase containing the hydrophobic amino acid and a bulking agent, if present.

21. (canceled)

22. A method according to claim 17, wherein the emulsion to be spray-dried will have a total powder mass of components (a) to (d) of between about 0.1 g and 10 g per 100 mL emulsion.

23-28. (canceled)
